(19) United States
(12) Patent Application Publication
(10) Pub. No.: US 2026/0325461 A1
(43) Pub. Date: Oct. 22, 2026

(54) NUTRITION RELATED SWELLABLE ITEM

(71) Applicant: GLYCEMIC SHIELD LTD., Kfar Mordechai (IL)

(72) Inventors: Amir Noy, Kfar Mordechai (IL); Yotem Levin, NESS TZIONA (IL)

(73) Assignee: GLYCEMIC SHIELD LTD., Kfar Mordechai (IL)

(21) Appl. No.: 18/994,044

(22) PCT Filed: Jul. 10, 2023
(86) PCT No.: PCT/IB2023/057084

§ 371 Date: Jan. 13, 2025

Related U.S. Application Data
(60) Provisional application No. 63/359,893, filed on Jul. 11, 2022.

(51) Int. Cl. A61K 9/70; A61K 9/00; A61K 31/715

(52) U.S. Cl. CPC A61K 9/70; A61K 9/0065; A61K 31/715

(57) ABSTRACT

A swellable item, that includes (i) a dissolvable surrounding element that is dissolvable in gastro-intestinal fluids, and (ii) a structure that is poroelastic, exhibits a compression and expansion ratio that exceeds ten, and is configured to absorb dissolved nutrients and chyme particles when expanding at a presence of gastro-intestinal fluids and to maintain a majority of the dissolved nutrients and chyme particles until exiting the small intestine. The dissolvable surrounding element is configured to surround the structure and maintain the structure in a compressed form before being dissolved. The swellable item is safe for human consumption.

### CROSS REFERENCE

This application claims priority of U.S. provisional patent 63/359,893 filing date Jul. 11, 2022 which is incorporated herein in its entirety.

### BACKGROUND

There is a growing need to lose weight is a safe manner.

Obesity is a global epidemic. The World Health Organization (WHO) estimates that in 2016, more than 1.9 billion adults (or 1 in 3 worldwide!) were overweight, and of those, over 650 million were obese. The prevalence of obesity has increased dramatically in recent decades, from about 10% in 1975, to over 30% of adults worldwide (2016). Obesity is associated with a number of chronic diseases, including heart disease, stroke, type 2 diabetes, and cancer. It is also a major risk factor for premature death.

There are a number of factors that contribute to the global obesity epidemic. These include:
Increased calorie intake, especially from processed foods and sugary drinks.
Decreased physical activity and sedentary lifestyle.
Genetic factors and Certain medications.

As a major challenge for humanity, the weight loss market includes a diversity of solutions from mild and non-invasive to definitive, invasive and hazardous. However, none of these solutions have been able to dramatically curb the obesity epidemic.

The solutions are broadly segmented by product type and intervention. By product type, there are weight loss supplements, drugs, surgical solutions, and weight loss programs. The weight loss supplements segment stands out as being one of the most natural and safe of all weight loss solutions.

Weight loss drugs are very expensive, require prescription, and have significant side effects and compliance challenges. Side effects include indigestion, pancreatitis and hypoglycemia; Weight loss surgery is exercised almost only with morbid obesity patients due to its risk profile (including risk of death during surgery), invasiveness, long term side effects and detrimental effects on quality of life.

Dieting, an almost universal intervention, typically has poor adherence challenges that reduce its effectiveness in the long term and are expensive; invariably, most solutions above also interfere with the pleasure and satisfaction of normal uninhibited eating.

There appears to be a huge unmet need with safe, effective, compliant and affordable interventions that do not pose significant hazard to individuals and which are scalable.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 illustrates an example of a sponge like open cell porous thick filament fiber;

FIG. 2 illustrates an example of a sponge like open cell porous thick filament fiber after cutting and in an expanded state;

FIG. 3 illustrates an example of a sponge like open cell structure manufactured by extrusion foaming and particulate or polymer leaching;

FIG. 4 illustrates an example of a sponge like open cell structure manufactured by spinning (electro spinning, dry jet wet spinning or other methods of spinning) of non-porous thin filament fibers spun helically through a spinneret perpendicular to the drawing axis to create a “yarn like” tangle non-woven fabric;

FIG. 5 illustrates an example of a porous filament fibers spun (twisted) to a compressed yarn.

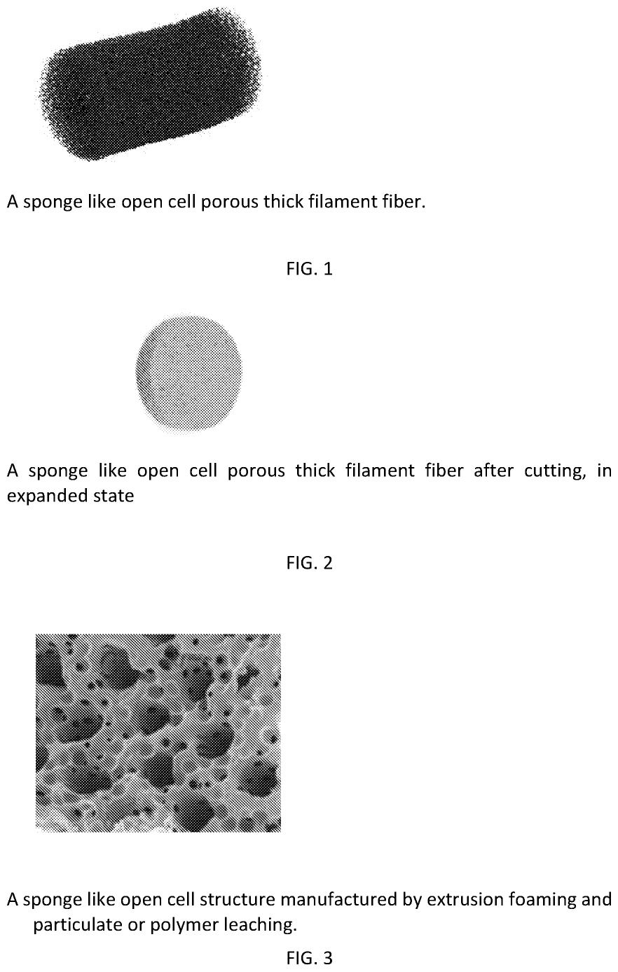

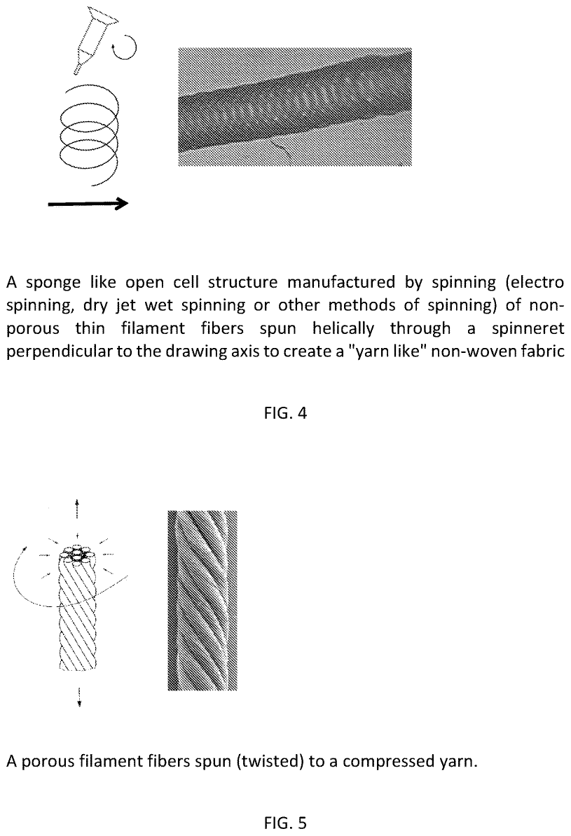

### DETAILED DESCRIPTION OF THE DRAWINGS

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

Because the illustrated embodiments of the present invention may for the most part, be implemented using chemical processes known to those skilled in the art, details will not be explained in any greater extent than that considered necessary as illustrated above, for the understanding and appreciation of the underlying concepts of the present invention and in order not to obfuscate or distract from the teachings of the present invention.

Any reference in the specification to a method should be applied mutatis mutandis to an swellable item manufactured by the method.

Any reference in the specification to an swellable item should be applied mutatis mutandis to a method for manufacturing the swellable item and/or should be applied mutatis mutandis for a method for administering the swellable item and/or should be applied mutatis mutandis to a method for consuming the swellable item.

There is provided an orally swellable item that includes a dissolvable surrounding element that is dissolvable in gastro-intestinal fluids and a poroelastic structure (which is highly porous and elastic), held compressed by the dissolvable surrounding and exhibits a compression ratio that exceeds ten.

The terms “compression ration”, “expansion ratio” and “compression and expansion ratio” refer to a relationship between a volume of a structure when expanded to the volume of the structure when compressed. The expanded volume exceeds the compressed volume.

The swellable item is configured to the following:
a. Transfer from the mouth, through the esophagus to the stomach while the structure is in compressed state.
b. The structure expands to its original size, in the stomach or in the duodenum, after the surrounding dissolves, exposed to gastro-intestinal fluids.
c. The structure absorbs gastro-intestinal fluids with dissolved nutrients and chyme particles while expanding.
d. The expanded structure maintains the majority of the gastro-intestinal fluids with dissolved nutrients and chyme particles while transferring through the small intestine, until exiting the ileocecal valve.
e. The structure releases most of the gastro-intestinal fluids while being mechanically compressed and/or disintegrated (by bacterial fermentation) in the colon.

The dissolvable surrounding element is configured to surround the structure and maintain the structure in a compressed form before being dissolved.

The swellable item is safe for human consumption. For example—the swellable item may be formed solely from Generally Recognized as Safe (GRAS) materials—as defined Under sections 201(s) and 409 of the Federal Food, Drug, and Cosmetic Act.

According to an embodiment, the poroelastic structure is made of cellulose.

According to an embodiment, the poroelastic structure is made of regenerated cellulose.

According to an embodiment, the poroelastic structure includes cellulose and a plasticizer.

According to an embodiment, the poroelastic structure includes cellulose and an additive that has a higher bacterial fermentability than the cellulose.

Cellulose mechanical characteristics are defined by both its average degree of polymerization and its crystallinity. The higher the average degree of polymerization (which is usually in the range of 20-20,000) and the higher the crystallinity index (α values which are usually in the range of 20-95%) the higher is the tensile strength and the Young's modulus of the fibers. But on the other hand, also the bacterial fermentation is lower due to lower enzymatic accessibility to the fiber. Thus, the cellulose can include a blend of high average degree of polymerization (e.g. 100-1500) and/or higher crystallinity index (e.g. α>30%) cellulose for good mechanical charasteristics and low average degree of polymerization (e.g. <100) and/or low crystallinity (e.g. α<30%) cellulose for good bacterial fermentation and dismantling.

According to an embodiment, the poroelastic structure is configured to absorb specific materials through its building materials or coating, as an example, it can be oleophylic in order to enhance fatty acid absorption.

According to an embodiment, the swellable item is further integrated or impregnated or combined or co-administered with edible food coloring and or food flavors and/or edible fragrances to improve taste, smell or appearance.

According to an embodiment, the porosity of the poroelastic structure exceeds ninety eight percent.

According to an embodiment, the swellable item is a powder particle (multi-particulate).

According to an embodiment, the swellable item is a powder particle having a length that ranges between 100 and 700 microns.

According to an embodiment, the swellable item is a capsule, encapsulating multiple such poroelastic structures in compressed state.

According to an embodiment, a volume of the daily orally delivered amount of swellable items will not exceed few tens of cubic centimeters, and According to an embodiment, the volume of the daily swelled amount of poroelastic structures when uncompressed will be few hundreds cubic centimeters.

According to an embodiment, a diameter of the expanded poroelastic structure does not exceed three millimeters and a length of the expanded poroelastic structure does not exceed ten millimeters.

According to an embodiment, the poroelastic structure is configured to withstand one cycle of full compression to the swellable item and full expansion after the surrounding dissolves, and also thousands of partial compression and expansion cycles within the small intestine.

According to an embodiment, the poroelastic structure is configured to be dismantled in a colon.

According to an embodiment, the structure includes multiple groups of pores, According to an embodiment, pores of each group of pores are in fluid communication with each other.

According to an embodiment, the poroelastic structure is a spongelike structure element of an interconnected pores network type (“open cell”: fluid flow is possible to penetrate among the pores and to and from the outside).

According to an embodiment, the structure includes multiple non-porous fibers that are spun to provide a poroelastic yarn.

According to an embodiment, the poroelastic structure is manufactured by a manufacturing process that includes generating a structure that includes fully enclosed pores (“closed cell”); and forming holes that link the pores among themselves and to the outside (“open cell”).

Various manufacturing processes are listed below.

There is provided swellable item that may include a swellable “sponge like” structure (“open cell” porous structure), which is swallowed while in compressed state within an outer case or coating and reaches its target expansion location (the stomach or the upper small intestine—duodenum or Jejunum), captures (absorbs) gastro-intestinal fluids with dissolved nutrients and chyme particles while expanding (before most of the nutrients absorption in the small intestine takes place), move along the gastro-intestinal tract transferring most of these materials while in expanded state throughout the small intestine to the colon and either transfers it outside the body or releases most of the trapped liquid and materials within the colon (with or without poroelastic structure full or partial dismantling in the colon).

The mixture of gastro-intestinal fluids and chyme can be partially in the form of a solution (dissolved materials, e.g. nutrients such as carbohydrates), a mixture of the fluids with small (up to 1 mm diameter) non-soluble particles or emulsion (e.g. fatty acid micelles).

In order to have a significant impact on the daily nutrition, the total volume of absorbed chyme should be in the order of at least several 100's of CC, while a reasonable volume for swallowing such swellable items is few CC to very few 10's of CC.

Thus, in order to achieve large absorbed volume with small volume of swallowed items, the poroelastic structure should have very high porosity (e.g. >98%) and very high compression ratio (e.g. >95%).

Gastric motility is characterized by relatively weak pressure waves in the upper stomach (<20 mmHg) and relatively strong and short (several seconds) pressure waves (40−>100 mmHg) in the distal antrum and pylorus. Small bowel motility is characterized by medium pressure waves (both peristalsis and segmental, <25mmHg), short (several seconds) with maximal frequency of 8-12 cycles per minute.

Colon motility is characterized by stronger pressure waves (50-100 mmHg) which are short (several seconds) in the proximal colon but much longer (up to almost static) in the distal colon.

The poroelastic structure should be elastic enough (not brittle) to withstand at least one cycle of compressing to its compressed packed state and decompressing to its expanded state in the gastro-intestinal tract.

It should also withstand the compression cycles within the gastro-intestinal tract (stomach and small intestine) without releasing too much of the trapped fluids and chyme particles, until entering the colon, there it should be compressed and/or disintegrated.

The way to achieve these two requirements is by having a relatively elastic structure, in which the cells' openings in the porous structure (holes in the pores' walls, connecting pores) are relatively small, limiting high frequency fluids motion between adjacent pores, allowing only slow filling or emptying. This way the poroelastic structure will withstand sharp compression forces as exist in the small bowel (with compression of <5%), but will be fully compressed in the distal colon.

The poroelastic structure can be dismantled within the colon (e.g. through degradation by bacteria fermentation), and/or compressed by the pressures in the colon to release most of the trapped fluids and particles, since there is no significant nutrients absorption at this stage.

It is preferred that all the materials of which the poroelastic structure are built from should be generally recognized as safe and permitted to be used as food, food supplements or dietary supplements, no new dietary ingredients (e.g. regenerated cellulose).

Different swellable items can be used for different populations (e.g. adults vs. children), for example: different delivering options (multiparticulates Vs. capsules), different poroelastic structure dimensions (e.g. 1 mm Vs. 3 mm diameter in expanded state) etc.

### Expanding Location

Expanding in the small intestine (as opposing to expanding in the stomach) has several advantages: (i) gastric fluids low acidity (after neutralizing in the duodenum), so release of some of the fluids further in the small intestine or the colon due to external pressure will not cause any acid burns, (ii) not accumulating in the stomach, which could have negative impacts such as stomach pain, nausea and vomiting, and (iii) lower viscosity fluids (easier absorption in the poroelastic structure).

The major disadvantage is the lower nutrients concentration in the solution/mixture, which leads to lower efficacy (amount of nutrients trapped and passed through the gastro-intestinal tract un-digested).

On the other hand, expanding within the stomach can also improve the satiety, especially if the expanded poroelastic structures are too big to exit the pyloric sphincter during standard digestion so it accumulates and released to the small intestine mostly during the interdigestive migrating motor complex, which means that the mass of expanded poroelastic structures remains in the stomach for a longer period of time.

It can be that a portion of the poroelastic structures will expand in the stomach while the remaining will expand in the small intestine.

Due to the high pressure waves in the distal antrum, pylorus and proximal duodenum, even if the poroelastic structure will expand in the stomach, it will probably loose most of the liquid during the transfer to the duodenum and will fill again in the proximal duodenum.

### Dimension Requirements

The dimension requirements can be on the swellable items (before surrounding dissolving and poroelastic structures expanding) as well as on the expanded poroelastic structures.

The requirements are for the following: (i) delivering (swallowing), (ii) Passing from the stomach to the small intestine (through the pyloric sphincter), and (iii) Passing along the small intestine and through the ileocecal valve to the colon.

There are two major options for delivering (swallowing) (i) Pills (capsules): In order to be swallowed as a capsules, the diameter of each ingested capsule should be <8 mm, and the length <20 mm, the volume is in the range of 0.5-1 CC. The main drawback is the large number of such capsules required to deliver large volumes and the fact that many people (especially young) cannot or dislike swallowing large pills, (ii) Multiparticulates: A large number of small particles, having dimensions in the 100's of microns range (e.g. 200-500 μm), delivered as a “powder” and drunk mixed with water. This delivery option is much easier for large volumes (e.g. 10's of CC), replacing 10's of capsules. The main drawback is the grittiness “sandy” feeling.

From the literature, In order to pass from the stomach through the pyloric sphincter to the small intestine in parallel to the chyme during the postprandial period, particles should be up to 2 mm in diameter and 5-10 mm length (depending on its stiffness, elastic structures can be somewhat larger).

Larger particles would remain in the stomach during the postprandial period and be mostly transferred over the course of the interdigestive period, during phase three of the mmc (migrating motor complex) cycle, after most of the fluids and chyme particles have already left the stomach. It can take more than one mmc cycle before large particles (e.g. 10-20 mm diameter) are transferred out from the stomach.

Poroelastic structure that should expand in the stomach can be up to 2-3 mm diameter in expanded state, in which case it would transfer to the small intestine during the postprandial period, or larger than this and accumulate in the stomach until the mmc cycle, causing longer satiety period but with the risk of over accumulation in the stomach.

Poroelastic structure that should expand in the small intestine should be up to 2-3 mm diameter in compressed state, in order to pass to the small intestine during the postprandial period and capture nutrients dissolved in the gastric fluids.

Passing along the small intestine (and through the ileocecal valve to the colon) requires a diameter of <20 mm. In order to avoid direct pressure on the expanded poroelastic structure during the pass within the small intestine (by moving a side), small poroelastic structures are preferred (up to few millimeters size).

For multiparticulates delivery option, since the requirements for the multiparticulates dimensions are much harsher (each particle dimensions <1 mm), each particle will easily pass through the pyloric sphincter during digestive period in compressed (and probably also expanded) state.

### Manufacturing process options

The manufacturing process for these “sponge like” open cell porous structure can be based on a wide variety of known methods, either for textile fibers and non-woven fabrics manufacturing (e.g. cellulose regeneration for fibers such as rayon fibers), filtration polymer membrane manufacturing (e.g. casting plus particulate leaching), tissue engineering/polymer scaffold manufacturing (e.g. extrusion foaming followed by porogen leaching) or even aerogel manufacturing (e.g. polymer dissolving, solvent exchange and freeze drying).

The manufacturing process can start with a solution (polymer dissolved in a solvent), a molten or softened thermoplastic polymer or a liquid containing monomers or non crosslinked polymer that will later on precipitate or coagulate into a polymeric structure.

These methods include for example: (i) thermoplastic foaming (either extrusion or injection molding), creating mostly closed cell foam, followed by opening holes in the closed cells to make an open cell foam structure, e.g. by particulate leaching—adding a soluble material, as particles, that will later on be dissolved and create “holes” in the foam bubbles walls, or combustion or chemical reticulation which removes the thin “bubble” (closed cell) walls. (ii) Phase separation (e.g. mixing a polymer-solvent solution with non-solvent). (iii) Emulsion templating (e.g. PolyHIPE). (iv) Porogen/particulate leaching (e.g. table salt or calcium carbonate). (v) Polymer leaching (e.g. water soluble polymer such as PEO). (vi) Freeze drying. (v) Spinning (e.g. dry jet wet spinning, solution blow spinning, wet spinning, electro-spinning) followed by collection as a non-woven structure (e.g. yarns). (vi) Removal of molecule groups from the polymer side chains (e.g. polymer deacetylation)

The different technics create different pores size range, 10's to 100's of microns by foaming or particulate leaching, 100's of nm to microns by polymer leaching and nm by removal of polymer side chains.

In an embodiment, a manufacturing process produces an “endless”, “sponge” like, poroelastic thick yarn (few millimeters diameter in non-compressed state) which has all the required mechanical and physical properties. This yarn is then twisted (spinned) in order to be compressed, coated in order to hold it in compressed state and to withstand gastro-intestinal conditions until required expansion and cut to appropriate length.

Although the “endless” yarns can be drawn through all production stages (from fiber creation until cutting), it could be beneficial to roll “endless” filament fibers after process steps and un-roll in proceeding process stages. One benefit can be load balancing of manufacturing processes, using several such fibers in parallel for slow processes (e.g. saponification). Another benefit could be performing different manufacturing stages at different locations (e.g. by sub-contractors).

In an embodiment, the manufacturing process starts with extrusion foaming of a cellulose thermoplastic derivative (e.g. ester derivative such as cellulose acetate or cellulose acetate butyrate, viscose: cellulose xanthate, cellulose propionate, ether derivative such as ethylcellulose, cellulose nitrite etc.). The extrusion is from a 200-300 μm diameter die into an “endless” closed cell porous single filament fiber yarn with a diameter of 2-3 mm.

The foaming can be done with the help of chemical or physical blowing agents, e.g. water, SC—CO2, N2, isobutene etc. Better volume expansion ratio is achieved with larger blowing agent molecules (lower diffusion) and lower process temperatures.

After gas foaming, even if the opening of the closed cells is done at very high yield, some pores will be still closed, containing gas, forming a kind of a spring. This is the reason that also the physical or chemical blowing agent should be generally recognized as safe (eliminating the use of most hydro fluoro carbons and such).

Other materials such as nucleating agents can be added to improve the foaming uniformity, pores size and the volume expansion ratio.

In order to have good thermoplastic behavior, plus good mechanical properties, a plasticizer can be added (e.g. glycerol, glycerol tri-acetate etc.).

Although good cellulose degradation in the colon (poroelastic structure dismantle, mixture/solution release) can be achieved due to the combination of the use of pure cellulose (derivatives low degree of substitution, after regeneration, will improve the enzymatic degradation) and large surface area, it could be also beneficial to add additives (with low solubility and high fermentability, e.g. xanthan gum or lower average degree of polymerization and/or lower crystallinity cellulose).

The yarns will be drawn and dipped in baths for further reticulation and/or leaching (opening the pores into “open cell” form and enlarging the porosity).

After opening the pores, the cellulose can be regenerated (e.g. by saponification of ester derivatives such as cellulose acetate, for example by dipping in a solution of NaOH/H2O, NaOH/Ethanol or NaOMe/MeOH, similar to saponification of acetate rayon fibers to rayon as done in the case of Fortisan fibers, described in U.S. Pat. No. 2,053,766).

Cellulose regeneration (e.g. deacetylation of cellulose acetate) will also lower the poroelastic structure mass by ~40-50% and create nano-pores in the poroelastic structure. This should also improve poroelastic structure dismantle in the colon by enlarging the poroelastic structure's surface area contacting fermenting bacteria. This can also increase the cellulose poroelastic structure's modulus due to the hydrogen bonds between cellulose molecules, formed after de-acetylation.

Afterwards it will be spun for twisting, either as single twisted fiber yarn or as multiple fibers bundle (in order to squeeze out the air and compress the yarn, as well as pack several such filament fibers together). The yarn spinning can be done using ring spinning as well as rotor, air jet or other methods (based on standard textile yarn manufacturing or unique technics).

After spinning, the compressed yarn goes through a bath (or air spray coating) in order to be coated with a coating that will hold the yarn compressed until dissolving.

An example for such hard crystalline coating that can hold the twisted fibers (single or bundle) is similar to hard panning of sugar, polyol (e.g. sugar alcohols), sucrose esters etc., performed by coating with multi thin layers of a syrup which crystallizes. Another option is similar to soft panning, a syrup that would not crystallize but be utilized as an adhesive to a powder (e.g. confectionary sugar, sugar mixed with starch etc.) “dusted” afterwards.

Another coating can be applied for delayed expansion, either in acidic environment (stomach) or neutral environment (small intestine). An example for such coating that can be used as enteric (dissolving only in the neutral acidity small intestine) is stearic or palmitic fatty acid (also generally recognized as safe food).

The coatings for mechanically holding the compressed poroelastic structure and delayed expansion can be one or two different coatings. In the case of two separate coatings, the second coating can also be provided after cutting the yarn, since coating before cutting the fibers means that the fibers are not coated at the cut sides, but only around the cylindrical surface.

The last stage is cut the coated, compressed twisted yarn to required length (in the case of multiparticulates: 200-500 μm).

In an embodiment, the manufacturing process starts with extrusion (without foaming) of an endless solid (non-porous) thick (2-3 mm) yarn comprised of cellulose thermoplastic derivative combined with large amount of soluble particles.

The materials can be the same as in embodiment one, the main difference from embodiment one is that the extrusion is of a solid polymer thick fiber (through a 2-3 mm diameter die) and not a foamed thick fiber (that expands after extrusion through 200-300 μm diameter die).

The open cell porous structure is formed by porogen/particulate leaching within a bath filled with an appropriate solvent (e.g. water for water soluble particles).

All further process stages can be similar to embodiment one (e.g. regeneration, twisting, coating etc.).

In an embodiment, the manufacturing process starts with filament fibers spinning (e.g. solution blow spinning through spinnerets) of “endless” thin non-porous filament fibers which build a non-woven, tangled, “thick yarn like” poroelastic fabric.

An example for such a fabric creation is by spinning helical shapes downwards while the fabric is drawn to the side. The spinning can be using single or multiple nozzles. The helices are built in several layers and the outcome is an “endless” few hundred microns to few millimeters wide (about the helix diameter in one axis and the number of layers multiplied by the fiber diameter on the other axis, both perpendicular to the “endless” drawing axis).

The spinning (extrusion through narrow holes to create thin filament fibers) should not be confused with spinning already existing poroelastic endless yarn using a spindle for twisting and compressing.

The spinning can be of molten thermoplastic cellulose derivative, as well as dissolved cellulose derivative (e.g. viscose: cellulose xantate or dissolved cellulose di-acetate in acetone) or dissolved pure cellulose (e.g. lyocell process).

Each fiber can be 1-100 μm diameter; the fabric should be 2-3 mm diameter (can also be of rectangular shape, compressed to 200-300 μm diameter after twisting). The materials can be the same as in embodiment one, the main difference from embodiment one is that the extrusion is of solid polymer thin fibers and not a foamed thick fiber.

This structure is inherently “open cell” (the gaps between the thin fibers), so there is no need for particulate leaching or reticulation.

All further process stages can be similar to embodiment one (e.g. regeneration, twisting, coating etc.).

There is provided a method for weight reduction of a human, the method comprises swallowing a swellable item illustrated in the specification and/or drawings and/or claims. The human may be a human that suffers from excess weight and/or a patient that was diagnosed as suffering for excess weight.

There is provided a method for weight reduction of a human, the method comprises administering to a person a swellable item illustrated in the specification and/or drawings and/or claims. The human may be a human that suffers from excess weight and/or a patient that was diagnosed as suffering for excess weight.

There is provided a method for weight reduction of a animal, the method comprises swallowing a swellable item illustrated in the specification and/or drawings and/or claims.

There is provided a method for weight reduction of a animal, the method comprises administering to a person a swellable item illustrated in the specification and/or drawings and/or claims.

There is provided a method for manufacturing a swellable item illustrated in the specification and/or drawings and/or claims.

In the foregoing specification, the invention has been described with reference to specific examples of embodiments of the invention. It will, however, be evident that various modifications and changes may be made therein without departing from the broader spirit and scope of the invention as set forth in the appended claims.

Any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermedial components. Likewise, any two components so associated can also be viewed as being “operably connected,” or “operably coupled,” to each other to achieve the desired functionality.

Other modifications, variations and alternatives are also possible. The specifications and drawings are, accordingly, to be regarded in an illustrative rather than in a restrictive sense.

In the claims, any reference signs placed between parentheses shall not be construed as limiting the claim. The word ‘comprising’ does not exclude the presence of other elements or steps then those listed in a claim. Furthermore, the terms “a” or “an,” as used herein, are defined as one or more than one. Also, the use of introductory phrases such as “at least one” and “one or more” in the claims should not be construed to imply that the introduction of another claim element by the indefinite articles “a” or “an” limits any particular claim containing such introduced claim element to inventions containing only one such element, even when the same claim includes the introductory phrases “one or more” or “at least one” and indefinite articles such as “a” or “an.” The same holds true for the use of definite articles. Unless stated otherwise, terms such as “first” and “second” are used to arbitrarily distinguish between the elements such terms describe. Thus, these terms are not necessarily intended to indicate temporal or other prioritization of such elements. The mere fact that certain measures are recited in mutually different claims does not indicate that a combination of these measures cannot be used to advantage.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

We claim

1. A swellable item, comprising:
a dissolvable surrounding element that is dissolvable in gastro-intestinal fluids;
a structure that is poroelastic, exhibits a compression and expansion ratio that exceeds ten, and is configured to (a) absorb gastro intestinal fluids that comprise dissolved nutrients and chyme particles when expanding at a presence of the gastro intestinal fluids, and (b) maintain a majority of the dissolved nutrients and chyme particles until exiting the small intestine;
wherein the dissolvable surrounding element is configured to surround the structure and maintain the structure in a compressed form before being dissolved; and
wherein the swellable item is safe for human consumption.

2. The swellable item according to claim 1 wherein the dissolvable surrounding element is configured to be dissolved in the stomach.

3. The swellable item according to claim 1 wherein the dissolvable surrounding element is configured to be dissolved in the duodenum.

4. The swellable item according to claim 1, wherein the structure is made mainly of a non-soluble and non-digestible polysaccharide.

5. The swellable item according to claim 1, wherein the structure is made mainly of regenerated cellulose.

6. The swellable item according to claim 1, wherein the structure is made mainly of micro-crystalline cellulose.

7. The swellable item according to claim 1, wherein the structure comprises cellulose and a plasticizer.

8. The swellable item according to claim 1, wherein the structure comprises cellulose and an additive that has a higher fermentability than the cellulose.

9. The swellable item according to claim 1, wherein the structure comprises a blend of cellulose having average degree of polymerization that exceeds one hundred and cellulose having average degree of polymerization below one hundred.

10. The swellable item according to claim 1, wherein the structure comprises a blend of cellulose having a crystallinity index that exceeds thirty percent and cellulose having a crystallinity index that is below thirty percent.

11. The swellable item according to claim 1, wherein a porosity of the structure exceeds ninety eight percent.

12. The swellable item according to claim 1, wherein the swellable item is a powder particle.

13. The swellable item according to claim 1, wherein the swellable item is a powder particle having a length that ranges between 300 and 1500 microns.

14. The swellable item according to claim 1, wherein the swellable item is a powder particle having a length that ranges between 100 and 500 microns.

15. The swellable item according to claim 1, comprising multiple structures that are poroelastic, wherein the dissolvable surrounding element is configured to surround the multiple structures.

16. The swellable item according to claim 15, wherein the dissolvable surrounding element is a capsule.

17. The swellable item according to claim 16, wherein the capsule multiple structures that are poroelastic, wherein the dissolvable surrounding element is configured to surround the multiple structures, wherein a volume of the capsule does not exceed two cubic centimeters, and wherein an aggregate volume of the multiple structures when uncompressed exceeds ten cubic centimeters.

18. The swellable item according to claim 1, wherein a diameter of the structure when expanded does not exceed three millimeters and a length of the structure when expanded does not exceed ten millimeters.

19. The swellable item according to claim 1, wherein the structure is configured to withstand one cycle of full compression to the swellable item and full expansion after the surrounding dissolves, and also thousands of partial compression and expansion cycles within the small intestine.

20. The swellable item according to claim 1, wherein the structure exhibits a compressive yield strength that exceeds 40 mmHg, for a strain rate higher than 0.2 Hz.

21. The swellable item according to claim 15, wherein the structure exhibits a compressive young modulus that exceeds 400 mmHg, for a strain rate higher than 0.2 Hz.

22. The swellable item according to claim 1, wherein the structure exhibits a compressive quasi-static strength that is below 80 mmHg, for a strain rate that does not exceed 0.01 Hz.

23. The swellable item according to claim 1, wherein the structure exhibits an expansion constant pressure exceeds 10 mmHg.

24. The swellable item according to claim 1, wherein the structure exhibits a leakage between 1 Picoliter to 50 Picoliter per second under pressure between 10 mmHg to 100mmHg.

25. The swellable item according to claim 1, wherein the structure exhibits “leakage” between 0.1% of its volume per second to 5% of its volume per second under pressure between 10 mmHg to 100 mmHg.

26. The swellable item according to claim 1, wherein the structure is configured to be dismantled in a colon.

27. The swellable item according to claim 1, wherein the structure comprises multiple groups of pores, wherein pores of each group of pores are in fluid communication with each other; wherein each group of pores is in fluid communication with an exterior of the swellable item, following a dissolving of the dissolvable surrounding element.

28. The swellable item according to claim 1, wherein the structure is manufactured by a manufacturing process that comprises generating a closed cell porous structure; and forming holes between pores to create an open cell porous structure.

29. The swellable item according to claim 1, wherein the poroelastic structure comprises a tangle of non-porous fibers.

30. The swellable item according to claim 1, wherein the structure is a spongelike structure element of an interconnected pores network type.

31. The swellable item according to claim 1, wherein the structure is manufactured by a manufacturing process that comprises generating a structure that comprises multiple groups of pores; and forming holes that link pores of each group of pores to each other.

32. The swellable item according to claim 1, wherein the swellable item materials are all considered Generally Recognized As Safe (GRAS) by the FDA.

33. The swellable item according to claim 1 comprising at least one of (a) edible food coloring, (b) one or more food flavors, (c) one or more edible fragrances.

34. The swellable item according to claim 1 wherein the structure comprises coating that differs from the dissolvable surrounding element.

35. The swellable item according to claim 34, the coating is configured to provide material selective absorption by the structure.

36. The swellable item according to claim 1 wherein the structure comprises one or more materials for material selective absorption by the structure.

37. A swellable item, comprising: a dissolvable surrounding element that is dissolvable in gastro-intestinal fluids; and a poroelastic structure that is held compressed by the dissolvable surrounding element and exhibits a compression ratio that exceeds ten; wherein the swellable item is safe for human consumption.

38. The swellable item according to claim 37, wherein the poroelastic structure is made mainly of a non-soluble and non-digestible polysaccharide.

39. The swellable item according to claim 37, wherein the poroelastic structure is made mainly of regenerated cellulose.

40. The swellable item according to claim 37, wherein the poroelastic structure is made mainly of micro-crystalline cellulose.

41. The swellable item according to claim 37, wherein the poroelastic structure comprises cellulose and a plasticizer.

42. The swellable item according to claim 37, wherein the poroelastic structure comprises cellulose and an additive that has a higher fermentability than the cellulose 43. The swellable item according to claim 37, wherein the structure comprises a blend of cellulose having average degree of polymerization that exceeds 100 and cellulose having average degree of polymerization below 100.

44. The swellable item according to claim 37, wherein the structure comprises a blend of cellulose having a crystallinity index that exceeds thirty percent and cellulose having a crystallinity index that is below thirty percent.

45. The swellable item according to claim 37, wherein a porosity of the poroelastic structure exceeds ninety eight percent.

46. The swellable item according to claim 37, wherein the swellable item is a powder particle.

47. The swellable item according to claim 37, wherein the swellable item is a powder particle and the dissolvable surrounding is an encircling coating.

48. The swellable item according to claim 37, wherein the swellable item is a powder particle having a length that ranges between 300 and 1500 microns.

49. The swellable item according to claim 37, wherein the swellable item is a powder particle having a length that ranges between 100 and 500 microns.

50. The swellable item according to claim 37, comprising multiple poroelastic structures, wherein the dissolvable surrounding element is configured to surround the multiple poroelastic structures held in compressed state.

51. The swellable item according to claim 50, wherein the dissolvable surrounding element is a capsule.

52. The swellable item according to claim 37, wherein the diameter of the expanded poroelastic structure does not exceed three millimeters and the length of the expanded poroelastic structure does not exceed ten millimeters.

53. The swellable item according to claim 37, wherein the poroelastic structure is configured to withstand one cycle of full compression to the swellable item and full expansion after the surrounding dissolves, and also thousands of partial compression and expansion cycles.

54. The swellable item according to claim 37, wherein the poroelastic structure exhibits a compressive yield strength that exceeds 40 mmHg, for a strain rate higher than 0.2 Hz.

55. The swellable item according to claim 37, wherein the poroelastic structure exhibits a compressive young modulus that exceeds 400 mmHg, for a strain rate higher than 0.2 Hz.

56. The swellable item according to claim 37, wherein the poroelastic structure exhibits a compressive quasi-static strength that is below 80 mmHg, for a strain rate that does not exceed 0.01 Hz.

57. The swellable item according to claim 37, wherein the poroelastic structure exhibits an expansion constant pressure >10 mmHg.

58. The swellable item according to claim 37, wherein the poroelastic structure exhibits “leakage” between 1 Picoliter to 50 Picoliter per second under pressure between 10 mmHg to 100 mmHg.

59. The swellable item according to claim 37, wherein the poroelastic structure exhibits “leakage” between 0.1% of its volume per second to 5% of its volume per second under pressure between 10 mmHg to 100 mmHg.

60. The swellable item according to claim 37, wherein the poroelastic structure comprises a tangle of non-porous fibers.

61. The swellable item according to claim 37, wherein the swellable item materials are all considered Generally Recognized As Safe (GRAS) by the FDA.

62. The swellable item according to claim 37, wherein the poroelastic structure is a spongelike structure element of an interconnected pores network type (“open cell”).

63. The swellable item according to claim 37 wherein the poroelastic structure comprises coating that differs from the dissolvable surrounding element.

64. The swellable item according to claim 64, the coating is configured to provide material selective absorption by the poroelastic structure.

65. The swellable item according to claim 37, wherein the poroelastic structure comprises one or more materials for material selective absorption by the structure.

66. A method for weight reduction of a human, the method comprises swallowing a swellable item according to any claim of claims 1-65.

67. A method for weight reduction of a human, the method comprises administering to a person a swellable item according to any claim of claims 1-65.

68. A method for weight reduction of a animal, the method comprises swallowing a swellable item according to any claim of claims 1-65.

69. A method for weight reduction of a animal, the method comprises administering to a person a swellable item according to any claim of claims 1-65.

70. A method for manufacturing a swellable item according to any claim of claims 1-65.
